(19) United States
(12) Patent Application Publication
(10) Pub. No.: US 2024/0390586 A1
(43) Pub. Date: Nov. 28, 2024

(54) MEDICAMENT DELIVERY DEVICE SUB-ASSEMBLY AND CORRESPONDING METHOD OF ASSEMBLY

(71) Applicant: SHL MEDICAL AG, ZUG (CH)

(72) Inventors: Antonio Bendek, Wellington, FL (US); Sebastian Karlsson, Stigtomta (SE); Slobodan Stefanov, Deerfield Beach, FL (US)

(21) Appl. No.: 18/564,981

(22) PCT Filed: May 9, 2022
(86) PCT No.: PCT/EP2022/062462

§ 371 Date: May 6, 2024

Related U.S. Application Data
(60) Provisional application No. 63/195,220, filed on Jun. 1, 2021.

(30) Foreign Application Priority Data
Jun. 16, 2021 (EP) 21179804.6

(51) Int. Cl. A61M 5/24; A61M 5/31; A61M 5/32

(52) U.S. Cl. CPC A61M 5/2466; A61M 5/32; A61M 2005/3103; A61M 2205/0216; A61M 2207/00

(57) ABSTRACT

The present disclosure relates to a medicament delivery device sub-assembly having an injection needle assembly, a cartridge and a seal. The injection needle assembly has opposite distal and proximal ends, and has a retainer member configured to be connectable to a medicament container; a hub configured to be coaxially displaceable within the retainer member and provided with an injection needle having proximal and distal pointed ends; and a cap member interactively connected to both the hub and to the retainer member. A sealing member can be arranged between the cap member and the retainer member for preventing contamination of the injection needle before use. The seal seals a gap between the injection needle assembly and the cartridge. Methods of assembly are also described.

### TECHNICAL AREA

The present invention relates to medicament delivery device sub-assemblies and corresponding methods of assembly, and in particular to medicament delivery device sub-assemblies comprising a seal, a cartridge and an injection needle assembly.

### BACKGROUND OF INVENTION

Different types of injection needle assemblies have been developed during a number of years, where the aim is to have a more or less complete assembly that is attached to a medicament container. Regarding the aspect of attachment of injection needles to medicament containers, there is often a requirement that the rear end of the injection needle should protrude into the interior of the container.

This could be a drawback if the medicament reacts with the material of the needle when exposed for a period of time. In that respect it would be desirable to have the rear part of the needle outside the container until the injection is to be performed. On the other hand, this then requires that the rear end of the needle could be moved fairly easily into the container and also that the rear end of the needle is kept in a sterile environment until it is moved into the container.

A novel solution to these requirements is presented in the document WO 2009/150078. In '078, an injection needle assembly is disclosed provided with a retainer member attachable to a medicament container. The retainer member is arranged with a central passage, in which a hub is arranged. The hub holds an injection needle having a proximal and distal pointed end. Further a cap member is removably attached to the retainer member. In an initial position, both ends of the injection needle are protected by the assembly, keeping the injection needle sterile.

The assembly is designed such that when the cap member is removed in order to deliver a dose of medicament, the cap member holder is moved in a distal direction in relation to the retainer member such that the distal pointed end of the injection needle penetrates a membrane of the medicament container in a medicament delivery device to which the injection needle assembly is attached.

The assembly works very well in most instances and the injection needle is kept sterile. However, in some rare instances, there might be a risk that foreign matter may enter into the interior of the assembly through the interface between the cap member and the retainer member, in particular if there is some relative movement between the two parts. Therefore it would be advantageous if the risk of contaminating the injection needle is further reduced or minimized.

Some solutions of providing sealing between components of a needle assembly have been developed. For instance document WO 2010/094916 discloses a drug container and delivery mechanism having a needle hub with an injection needle provided with a proximal pointed end and a distal pointed end. A sealing element is arranged between the needle hub and a fastening component, intended to be connected to a medicament container. A further sealing element is arranged between the needle hub and a cap member.

When the assembly is to be used, the user manually removes the cap member, and then penetrates an injection site. It is only then that the distal end of the needle penetrates a covering membrane in order to obtain access to the medicament inside the container. This is a drawback in many instances and for many members that the passage for the medicament through the injection needle is created at the instance of the penetration. In many cases it is clearly desirable that a passage is created beforehand such that e.g. priming may be performed.

Another approach is provided in WO 2014/076225 which is herein incorporated by reference; the applicant has however realised that further improvements can be made.

### BRIEF DESCRIPTION OF INVENTION

In the present application, when the term “distal part/end” is used, this refers to the part/end of the assembly, or the parts/ends of the members thereof, which is/are located the furthest away from the medicament delivery site of the patient. Correspondingly, when the term “proximal part/end” is used, this refers to the part/end of the assembly, or the parts/ends of the members thereof, which, is/are located closest to the medicament delivery site of the patient.

The invention is defined by the claims, to which reference should now be made.

An aspect of the invention concerns a medicament delivery device sub-assembly comprising an injection needle assembly, a cartridge and a seal, the injection needle assembly having opposite distal and proximal ends, the injection needle assembly comprising: a retainer member configured to be connectable to a medicament container; a hub configured to be coaxially displaceable within the retainer member and provided with an injection needle having proximal and distal pointed ends; a cap member interactively connected to both the hub and to the retainer member; a sealing member arranged between said cap member and said retainer member for preventing contamination of said injection needle before use, wherein the seal seals a gap between the injection needle assembly and the cartridge. The seal can isolate a sterile volume between the cartridge and the injection needle assembly. In some embodiments, the seal is a separate component. Alternatively, the seal is attached to or an integral part of the cartridge. Alternatively, the seal is attached to or an integral part of the injection needle assembly.

Optionally, the seal is ring-shaped. Optionally, the seal comprises a central passage. Optionally, the seal is made of an elastic material. Optionally, the seal is made of a thermoplastic elastomer. Optionally, the seal is an O-ring. Optionally, the seal is a pad. Optionally, the seal is a distally extending extension of a transversal extending wall of the injection needle assembly, wherein the transversal extending wall is between the cartridge and the injection needle.

Optionally, the seal is in the gap between the injection needle assembly and one or more of a cylindrical portion of the cartridge, a shoulder of the cartridge, a neck of the cartridge, a head of the cartridge, a membrane of the cartridge and a crimp of the cartridge. Optionally, the seal is in the gap between the cartridge and one or more of a transversal extending wall of the injection needle assembly and a distal tubular part of the injection needle assembly.

According to a preferable solution, the sealing member comprises an elastic member, is generally ring-shaped and is attached to said retainer member. This is in order to increase the sealing properties of the sealing member, and maintain a sealing action even if there is some movement between the cap member and the retainer member.

According to one aspect of the invention and in order to provide a proper sealing action of the sealing member, said sealing member is arranged with a proximally directed contact surface, and said cap member is arranged with a distally directed contact surface.

According to another aspect of the invention and in order to further ascertain the sealing action between the cap member and the retainer member, said sealing member is arranged such in relation to said retainer member and said cap member that said sealing member is compressed when said cap member is attached to said retainer member. Thus the sealing member provides enhanced protection of the injection needle in that the risk of any foreign matter passing between the cap member and the retainer member is greatly reduced. Thereby it is ensured that the sterility is kept intact until the cap member is removed from the retainer member.

According to a further aspect of the invention, the distal pointed end is arranged to penetrate a membrane arranged at the proximal end of the medicament container.

According to yet another aspect of the invention, the hub comprises on its outer circumferential surface a distal engagement member and a proximal engagement member.

According to another aspect of the invention, the retainer member comprises a distal tubular part having a container attachment member, a proximal part having an outer engagement member on its outer circumferential surface, and a central passage having opposite distal and proximal ends and an inner engagement member on its inner circumferential surface adapted to receive the distal engagement member of the hub.

According to a further aspect of the invention, the cap member comprises on its inner circumferential surface a first engagement member arranged to cooperate with the proximal engagement member of the hub, and a second engagement member arranged to cooperate with the outer engagement member of the retainer member.

According to yet another aspect of the invention, the first engagement member of the cap member is arranged to cooperate with the proximal engagement member of the hub, such that the hub is allowed to be axially displaceable but prevented to be rotated in relation to the cap member.

According to another aspect of the invention, wherein the second engagement member of the cap member is arranged to cooperate with the outer engagement member of the retainer member such that the cap member is allowed to be axially displaceable in relation to the retainer member from a first position, in which the cap member is prevented to be removed from the retainer member, to a second position, in which the cap member is allowed to be removed from the retainer member.

According to a further aspect of the invention, wherein the inner engagement member of the retainer member is arranged to cooperate with the distal engagement member of the hub such that the hub is allowed to be coaxially displaceable in relation to the retainer member from a disconnected position in which the distal engagement member is positioned at the proximal end of the central passage to a connected position in which the distal engagement member is positioned at the distal end of the central passage.

According to yet another aspect of the invention, the hub is displaced from the disconnected to the connected position when the cap member is displaced from the first to the second position.

According to another aspect of the invention, the inner engagement member of the retainer member and the distal engagement member of the hub are guiding members, and the second engagement member of the cap member and the outer engagement member of the retainer member are also guiding members.

According to yet another aspect of the invention, the inner engagement member of the retainer member and the distal engagement member of the hub are threaded members; and the second engagement member of the cap member and the outer engagement member of the retainer member are threaded members.

According to a further aspect of the invention, a medicament delivery device comprises an injection needle assembly according to any of the preceding aspects of the invention. Optionally, the medicament delivery device is an autoinjector.

According to a further aspect of the invention, a method of assembly of a medicament delivery device sub-assembly (or of a medicament delivery device comprising the medicament delivery device sub-assembly) is provided, the method comprising the steps of: providing an injection needle assembly; providing a cartridge; and assembling the cartridge and the injection needle assembly together with a seal in a sterile environment, so that the seal isolates a sterile volume between the cartridge and the injection needle assembly. Preferably, the injection needle assembly is an injection needle assembly as described above.

These and other aspects of, and advantages with, the present invention will become apparent from the following detailed description of the invention and from the accompanying drawings.

### BRIEF DESCRIPTION OF DRAWINGS

In the following detailed description of the invention, reference will be made to the accompanying drawings, of which

FIG. 1 is a perspective view of an injection needle assembly according to the present invention,

FIG. 2 is an exploded view of the assembly of FIG. 1,

FIG. 3 is a cross-sectional view of the assembly of FIG. 1,

FIGS. 4-5 are detailed views of the assembly of FIG. 1,

FIG. 6 is a perspective view of the assembly of FIG. 1 in an operational state,

FIG. 7 is a cross-sectional view of a medicament delivery device sub-assembly comprising an injection needle assembly and a cartridge,

FIG. 8 shows a close-up of part of FIG. 7.

FIGS. 9, 10, 11 and 12 show different sealing options for the medicament delivery device sub-assembly of FIG. 7,

FIG. 13 is a cross-sectional view of another medicament delivery device sub-assembly comprising an injection needle assembly, a cartridge and a seal, and

FIG. 14 is a perspective view of the injection needle assembly shown in FIG. 13.

### DETAILED DESCRIPTION OF THE INVENTION

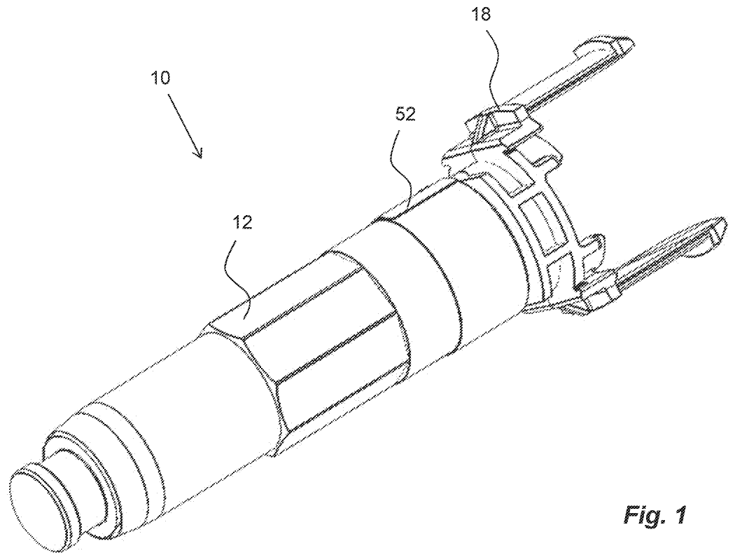

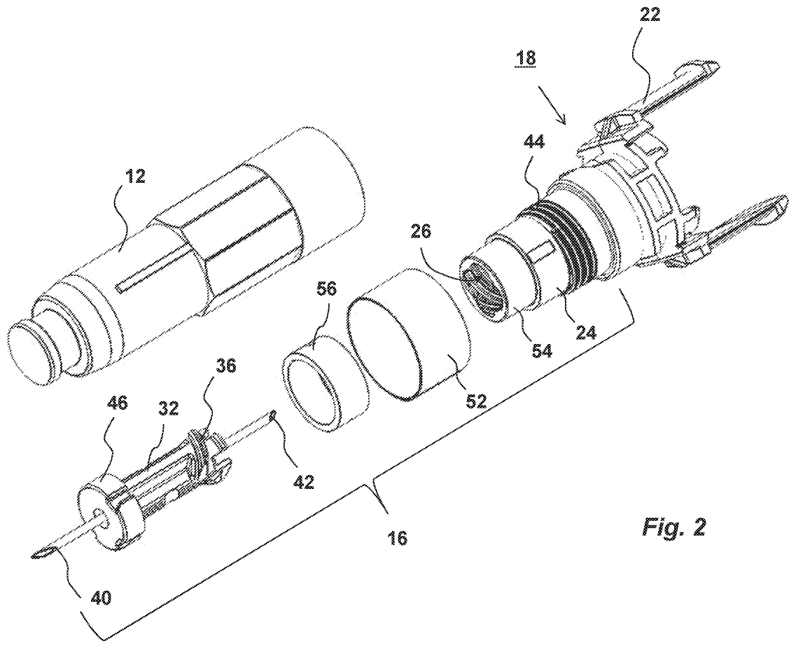

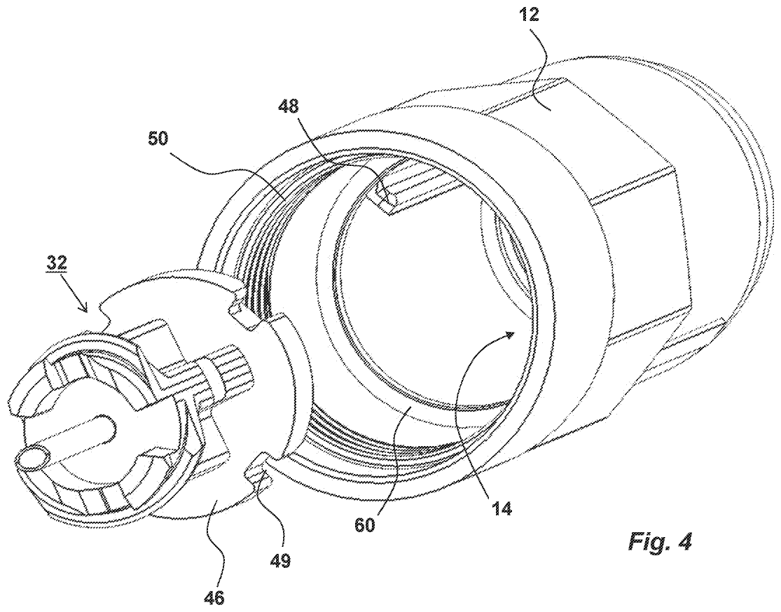

FIG. 1 shows an injection needle assembly 10. The injection needle assembly 10 has opposite distal and proximal ends. It comprises a retainer member 18 configured to be connected to a medicament container (not shown) in medicament delivery device; a hub 32 configured to be coaxially displaceable within the retainer member and provided with an injection needle 38 having proximal and distal pointed ends (40, 42); and a cap member 12 interactively connected to both the hub and to the retainer member. The distal end of the cap member is arranged with an opening 14, FIG. 4, accessing the interior of the cap member 12, wherein an injection part 16 of the needle assembly is enclosed, FIG. 2. The distal pointed end 42 of the injection needle is arranged to penetrate a membrane arranged at the proximal end of the medicament container. The injection needle assembly 10 further comprises a sealing member 56 arranged between said cap member 12 and said retainer member 18 for preventing contamination of said injection needle 38 before use.

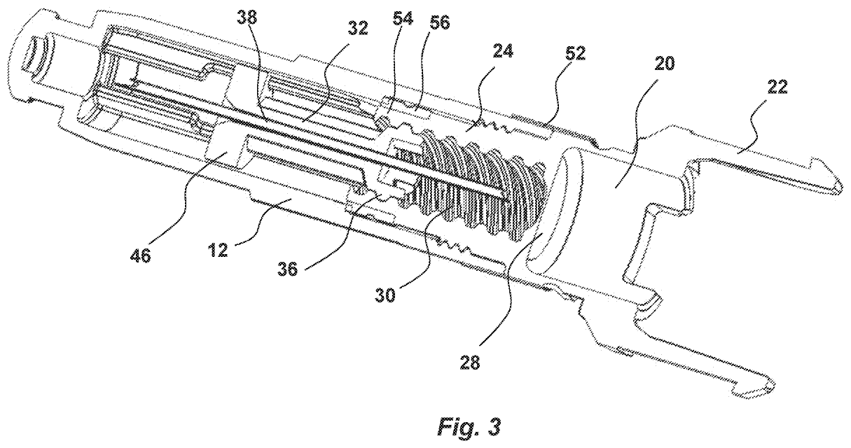

The retainer member 18 is arranged with a distal tubular part 20, FIG. 3, having a container attachment member into which a neck portion of a medicament container, such as a cartridge, may fit, and where the medicament container is intended to be attached either by threads, by gluing or by welding, for example. The retainer member 18 is further provided with distally extending fastening means 22 for attachment to a housing part of a medicament delivery device.

The retainer member 18 is further arranged with a proximal tubular part 24 having an outer engagement member 44 on its outer circumferential surface, and a central passage 26. Said central passage has opposite distal and proximal ends and an inner engagement member 30 on its inner circumferential surface. A transversal extending wall 28, FIG. 3, is separating the distal tubular part 20 and the central passage 26, as will be described below. In the embodiment shown in FIG. 3, the inner engagement member 30 of the passage 26 is arranged with threads 30. In a further embodiment, (not shown), the inner engagement member 30 of the passage 26 is arranged with guiding members e.g. ribs or grooves.

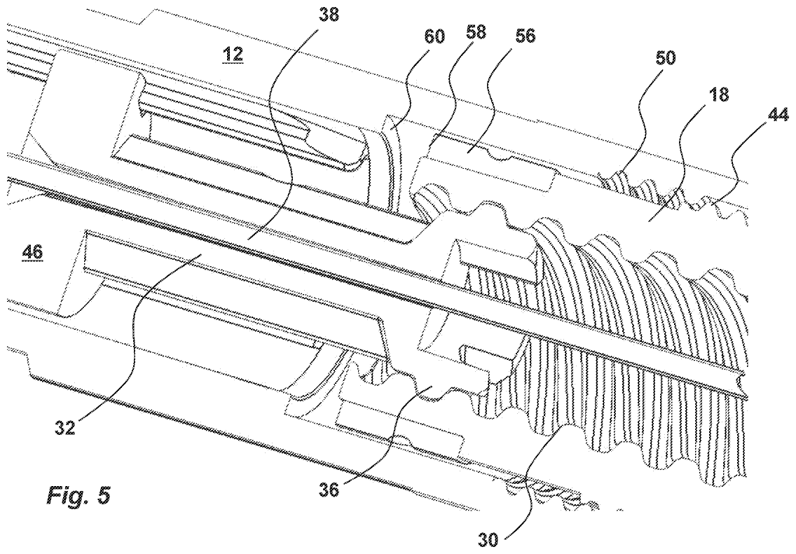

The hub 32 is a tubular shaped member, FIGS. 2 and 5, comprising on its outer circumferential surface a distal engagement member 36 and a proximal engagement member 46. In the embodiment shown in FIG. 3, the distal engagement member 36 of the hub 32 is a threaded member designed to interact with the threads 30 of the central passage 26. In a further embodiment, (not shown), the distal engagement member 36 of the hub 32 is a guiding member e.g. ribs or grooves designed to interact with the corresponding guiding member in the passage 26. The injection needle 38 is attached through the hub 32, having proximal and distal pointed ends (40, 42).

The cap member 12 is a tubular shaped member, FIG. 4, comprising on its inner circumferential surface a first engagement member 48 arranged to cooperate with the proximal engagement member 46 of the hub 32, and a second engagement member 50 arranged to cooperate with the outer engagement member 44 of the retainer member 18.

The proximal engagement member 46 of the injection hub 32, FIG. 4, is arranged with a number of longitudinally extending grooves or cut-outs 49 and the first engagement member 48 is a longitudinally extending rib. Each groove or cut-out 49 is intended to receive a longitudinally extending rib, such that a rotational lock is obtained between the cap member 12 and the hub 32, while allowing movement in the longitudinal direction between the two i.e. the hub 32 is allowed to be axially displaceable but prevented to be rotated in relation to the cap member 12.

The second engagement member 50 of the cap member 12 is arranged to cooperate with the outer engagement member 44 of the retainer member 18 such that the cap member 12 is allowed to be axially displaceable in relation to the retainer member from a first position, in which the cap member 12 is prevented to be removed from the retainer member, to a second position, in which the cap member 12 is allowed to be removed from the retainer member.

The inner engagement member 30 of the retainer member 18 is arranged to cooperate with the distal engagement member 36 of the hub 32 such that the hub 32 is allowed to be coaxially displaceable in relation to the retainer member 18 from a disconnected position in which the distal engagement member 36 is positioned at the proximal end of the central passage 26 to a connected position in which the distal engagement member 36 is positioned at the distal end of the central passage 26.

In the embodiment shown in FIG. 3, the second engagement member 50 of the cap member 12 and the outer engagement member 44 of the retainer member 18 are threaded members. The threads 44 are intended to cooperate with threads 50, FIG. 4, for releasibly attaching the cap member 12 to the retainer member 18. In a further embodiment, (not shown), the second engagement member 50 of the cap member 12 and the outer engagement member 44 of the retainer member 18 are guiding members e.g. ribs or grooves.

A tamper indication member 52, FIGS. 1 and 2, is arranged, comprising an adhesive label or sticker attached outside the interface between the cap member 12 and the retainer member 18.

Further, the retainer member 18 is arranged with a circumferentially recessed proximal portion 54, FIG. 2. This portion is arranged to accommodate the sealing member 56. In the embodiment shown, the sealing member is a ring-shaped member made of an elastic material, having a proximally facing circumferential surface 58. This surface 58 is intended to be in contact with a distally directed circumferential ledge 60, FIGS. 4 and 5, on the inner surface of the cap member 12. The sealing member 56, the recessed portion 54 and the circumferential ledge 60 are designed such that the sealing member 56 is compressed somewhat when the cap member 12 is attached to the retainer member 18. The sealing member 56 thus creates an effective seal between the cap member 12 and the retainer member 18, thereby preventing any contamination of the injection needle 38 before use of the needle assembly.

When the injection needle assembly is to be used, the cap member 12 is displaced from the first to the second position and because of the connection with the hub 32; the latter is also displaced from the disconnected to the connected position.

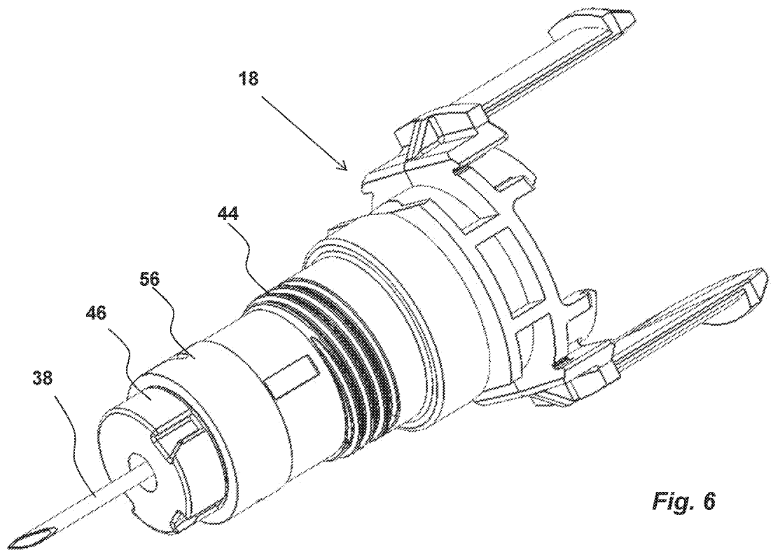

According to the embodiment shown in the figures, when the cap member 12 is turned, the hub 32 is also turned. However, the threads between the cap member 12 and the retainer member 18 have a different direction of the pitch than the threads between the hub 32 and the retainer member 18. Therefore, when the cap member 12 is turned for removing it from the retainer member 18 in the proximal direction, the turning of the injection hub 32 causes it to move in the distal direction into the retainer member 18, FIG. 6, whereby the pointed distal end 42 of the injection needle 38 penetrates the transversal extending wall 28 of the retainer member 18 as well as the membrane of the medicament container and due to the different direction of the pitch the cap member is screwed off the hub and may be removed, FIG. 6.

According to a further embodiment, when the cap member 12 is first axially displaced towards the distal end of the assembly and the turned in relation to the retainer member 18, the hub 32 is also is axially displaced towards the distal end of the assembly and turned in relation to the retainer member. For removing the cap member, the guiding members between the cap member 12 and the retainer member 18 are configured such that the cap member can only be axially displaced towards the distal end of the assembly, turned and then displaced towards the proximal end of the assembly. The guiding members between the hub 32 the retainer member 18 are configured such that the hub is can be axially displaced towards the distal end of the assembly and turned. Therefore, when the cap member 12 is pushed, turned and pulled for removing it from the retainer member 18, the axial displacement of the injection hub 32 causes it to move in the distal direction into the retainer member 18, whereby the pointed distal end 42 of the injection needle 38 penetrates the transversal extending wall 28 of the retainer member 18 as well as the membrane of the medicament container.

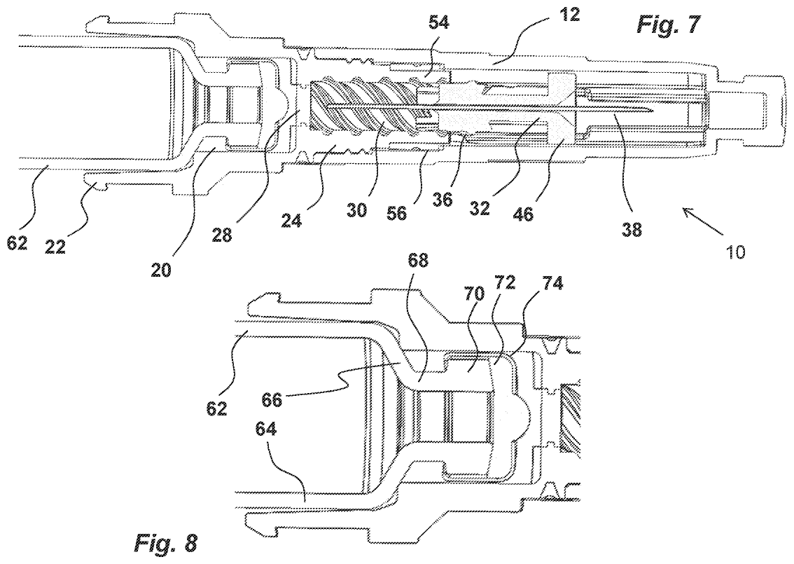

Another embodiment is shown in FIG. 7. The design is similar to the injection needle assembly 10 described above, and will not be fully described again in the interests of succinctness. In addition to the features already described above, a cartridge 62 is shown. FIG. 8 shows a close-up of part of FIG. 7. The cartridge 62 comprises a cylindrical portion 64, a shoulder 66, a neck 68, a head 70, a membrane 72 and a crimp 74 holding the membrane 72 onto the head 70. This particular shape of cartridge is shown as an example, but other cartridge shapes and types could also be used. For example, the crimp 74 is optional.

During assembly of a medicament delivery device, keeping the area between the cartridge and the injection needle assembly sterile can be beneficial. In particular, it can be beneficial if the path subsequently taken by the needle from the position shown in FIG. 7 to a position in which the needle penetrates the cartridge is sterile. One solution is to control the sterility of the manufacturing process during part or all of medicament delivery device assembly. However, this can be difficult and/or expensive, and it has been appreciated that it would be beneficial to minimise the number of assembly steps that need to be carried out in a sterile environment. Provision of a seal between the injection needle assembly 10 and the cartridge 62 can help with this, as the needle path can then be isolated once the injection needle assembly, the seal and the cartridge have been assembled together.

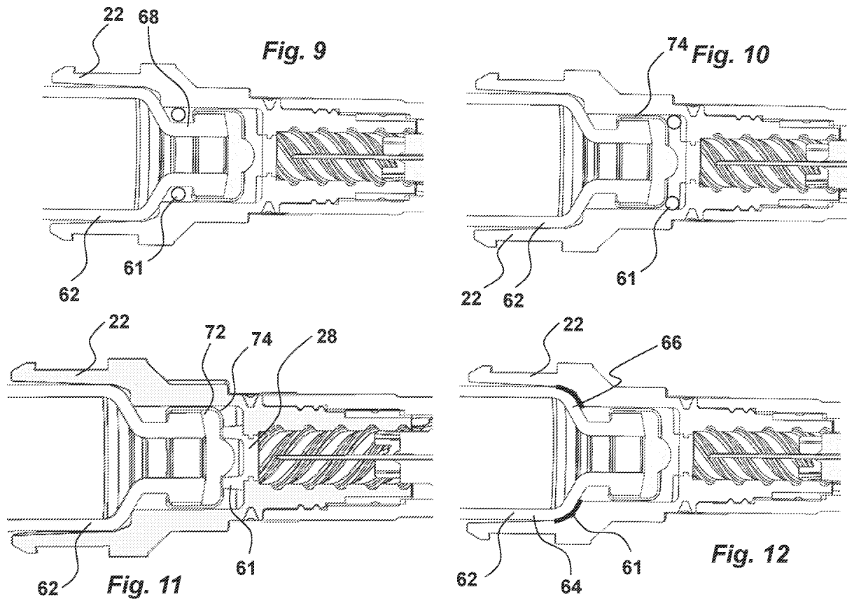

To this end, a seal (not shown in FIG. 7) can be provided in various places between the cartridge and the injection needle assembly. The seal is provided to seal the gap between the injection needle assembly and the cartridge. The seal can be various shapes and materials and can be provided in various locations, as will now be described. In some examples, the seal is attached to (or is an integral part of) the injection needle assembly, or is attached to (or is an integral part of) the cartridge, or is a standalone component. For example, a seal could be provided in the gap between the injection needle assembly 10 and one or more of the cylindrical portion 64, the shoulder 66, the neck 68, the head 70, the membrane 72 and the crimp 74 of the cartridge 62. In general, the gap is between the cartridge 62 and the distal end of the injection needle assembly 10, in particular between the cartridge 62 and the distal tubular part 20 of the injection needle assembly 10 or between the cartridge 62 and the transversal extending wall 28 of the injection needle assembly 10. In particular, the seal could be provided in the gap between the head 70, the membrane 72 or the crimp 74 of the cartridge 62 and the transversal extending wall 28 of the injection needle assembly 10 (see for example FIG. 11). In particular, the seal could be provided in the gap between the cylindrical portion 64, the shoulder 66, the neck 68, the head 70, the membrane 72 or the crimp 74 of the cartridge 62 and the distal tubular part 20 of the injection needle assembly 10 (see for example FIGS. 9 and 10).

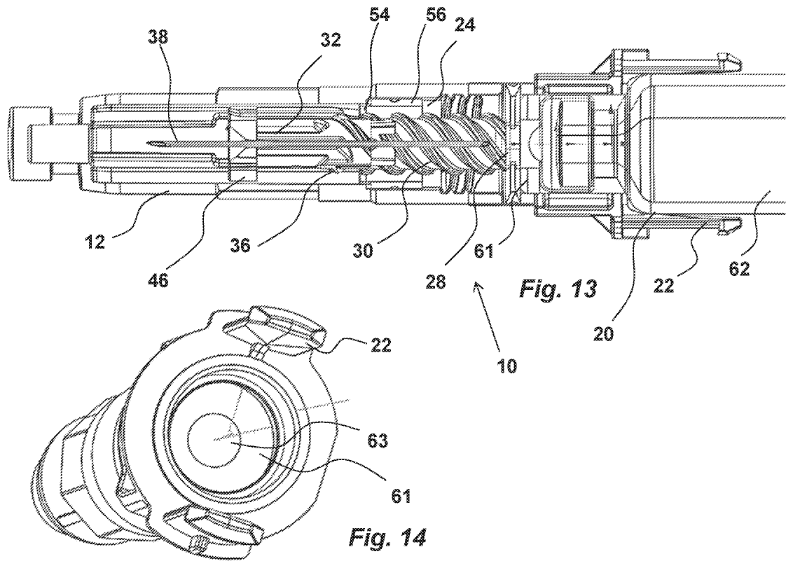

A non-exhaustive list of examples of a seal will now be described. FIGS. 9 to 12 show examples of a seal between the injection needle assembly 10 and the cartridge. In FIGS. 9 and 10, the seal is an O-ring. In FIG. 9, the O-ring extends between the neck of the cartridge and the distal tubular part 20 of the injection needle assembly 10. In FIG. 10, the O-ring extends between the crimp of the cartridge and the distal tubular part 20 of the injection needle assembly 10. In FIG. 11, the seal is a distally extending extension of the transversal extending wall 28 of the injection needle assembly 10, and extends between the crimp of the cartridge and the distal tubular part 20 of the injection needle assembly 10. In FIG. 12, the seal is a pad (for example a flexible pad such as a TPE (thermoplastic elastomer) pad) between the shoulder of the cartridge and the distal tubular part 20 of the injection needle assembly 10. A further example will now be described with reference to FIGS. 13 and 14. Much of the design is similar to those described above, and will not be described again in the interests of succinctness. In this example, the seal is a ring extending between the proximal-most end of the cartridge (the head in this case) and the injection needle assembly 10; more specifically, the seal extends between the crimp of the cartridge and the distal tubular part 20 of the injection needle assembly 10.

Examples of the seal as an O-ring, a ring, a pad, and an extension of another part of the injection needle assembly 10 have been shown, and each of these examples could be provided in other parts of the gap between the cartridge and the injection needle assembly 10 than where they are shown. In general, the seal may be flexible or rigid. Flexible seals may be preferable (particularly seals that are elastic) as they can provide greater tolerance to manufacturing inaccuracies and/or a more complete seal. The seal could be made of various materials, including elastic materials, for example TPE, rubber, or another plastic.

Preferably, the seal comprises a central passage such as the central passage 63, as shown in FIG. 14. This allows the needle to pass through the central passage during use. The provision of a central passage is optional, though, as the needle could alternatively pierce the seal during use rather than extending through the central passage.

The examples described above each provide a single seal. However, two or more seals (either of the same type or different types) could be provided to further improve the seal.

A method of assembly of a sub-assembly such as that shown in FIG. 13 will now be described. Firstly, an injection needle assembly 10 as described above is provided along with a seal and a cartridge. Optionally, part or all of one or more of the injection needle assembly, the seal and the cartridge are sterilised, for example by a surface sterilisation technique such as e-beam (electron-beam sterilisation)—for example, the inside of the distal tubular part 20 of the injection needle assembly 10 may be sterilised in this step. This sterilisation step may be needed if components have been kept in a non-sterile environment between manufacture and assembly. Then, in a sterile environment, the sub-assembly is assembled by assembling the cartridge and the injection needle assembly together with the seal. A complete medicament delivery device including this sub-assembly could then be assembled. Although it is not necessarily needed to maintain sterility of the needle path, further medicament delivery device assembly steps could also be carried out in a sterile environment.

The cartridge and injection needle assembly could be attached to one another, for example by a friction fit (for example by friction between the cartridge and either the distally extending fastening means 22 and/or the distal tubular part 20) or a snap fit, although alternatively the cartridge and injection needle assembly are not attached to one another and are held in place relative to one another by other medicament delivery device components.

The seal 61 can either be an integral part of the injection needle assembly (for example a moulded feature of a component of the injection needle assembly—this could be the case in the example in FIG. 11, for example) or can be a separate component. When the seal 61 is a separate component, the seal 61 can be attached to the injection needle assembly or to the cartridge, or can alternatively just be arranged in place in the injection needle assembly or on the cartridge (without attachment) during assembly. The exact step or steps required to assemble the cartridge and the injection needle assembly together with the seal will therefore depend on whether the seal is attached to (or part of) the cartridge, attached to (or part of) the injection needle assembly, or a standalone component. For example, if the seal is a separate component like the O-ring shown in FIG. 9, one method of assembly would be to first arrange the seal around the neck of the cartridge, and then place the injection needle assembly 10 onto the cartridge. As another example, if the seal is a separate component like the O-ring shown in FIG. 10, one method of assembly would be to first insert the seal into the distal tubular part 20 of the injection needle assembly 10, and then insert the cartridge into the distal tubular part 20 of the injection needle assembly 10. As a third example, if the seal is a part of the injection needle assembly 10 such as the option shown in FIG. 11, one method of assembly would be to insert the cartridge into the distal tubular part 20 of the injection needle assembly 10. As a fourth example, if the seal is a part of the cartridge as shown in FIG. 12, one method of assembly would be to insert the cartridge into the distal tubular part 20 of the injection needle assembly 10. For all of these examples, however, other assembly options are available, for example depending on the specific shapes of the components and on the type of assembly line being used (e.g. a manual, semi-automatic or automatic assembly line).

In general, the concepts described herein could be provided in various medicament delivery devices, such as autoinjectors or pen injectors, for example.

It is to be understood that the embodiments described above and shown in the drawings are to be regarded only as non-limiting examples of the invention that may be modified in many ways within the scope of the patent claims.

Some aspects of the invention are described in the clauses below.
1. A medicament delivery device sub-assembly comprising an injection needle assembly (10), a cartridge (62) and a seal (61), the injection needle assembly (10) having opposite distal and proximal ends, the injection needle assembly (10) comprising:
   a retainer member (18) configured to be connectable to a medicament container;
   a hub (32) configured to be coaxially displaceable within the retainer member and provided with an injection needle (38) having proximal and distal pointed ends (40; 42);
   a cap member (12) interactively connected to both the hub and to the retainer member; and
   a sealing member (56) arranged between said cap member (12) and said retainer member (18) for preventing contamination of said injection needle (38) before use,
   wherein the seal (61) seals a gap between the injection needle assembly (10) and the cartridge (62).
2. The medicament delivery device sub-assembly according to clause 1, wherein the seal (61) is ring-shaped.
3. The medicament delivery device sub-assembly according to clause 1 or 2, wherein the seal (61) comprises a central passage (63).
4. The medicament delivery device sub-assembly according to any previous clause, wherein the seal (61) is made of an elastic material.
5. The medicament delivery device sub-assembly according to any previous clause, wherein the seal (61) is made of a thermoplastic elastomer.
6. The medicament delivery device sub-assembly according to any previous clause, wherein the seal (61) is an O-ring.
7. The medicament delivery device sub-assembly according to any of clauses 1 to 5, wherein the seal (61) is a pad.
8. The medicament delivery device sub-assembly according to any previous clause, wherein the seal (61) is a distally extending extension of a transversal extending wall (28) of the injection needle assembly (10), wherein the transversal extending wall (28) is between the cartridge (61) and the injection needle (38).
9. The medicament delivery device sub-assembly according to any previous clause, wherein the seal (61) is in the gap between the injection needle assembly (10) and one or more of a cylindrical portion (64) of the cartridge (62), a shoulder (66) of the cartridge (62), a neck (68) of the cartridge (62), a head (70) of the cartridge (62), a membrane (72) of the cartridge (62) and a crimp (74) of the cartridge (62).
10. The medicament delivery device sub-assembly according to any previous clause, wherein the seal (61) is in the gap between the cartridge (62) and one or more of a transversal extending wall (28) of the injection needle assembly (10) and a distal tubular part (20) of the injection needle assembly (10).
11. The medicament delivery device sub-assembly according to any of clauses 1 to 10, wherein said sealing member (56) comprises an elastic member.
12. The medicament delivery device sub-assembly according to any of clauses 1 to 11, wherein said sealing member (56) is generally ring-shaped.
13. The medicament delivery device sub-assembly according to clause 12, wherein said sealing member (56) is attached to said retainer member (18).
14. The medicament delivery device sub-assembly according to clause 13, wherein said sealing member (56) is arranged with a proximally directed contact surface (58), and wherein said cap member (12) is arranged with a distally directed contact surface (60).
15. The medicament delivery device sub-assembly according to clause 14, wherein said sealing member (56) is arranged such in relation to said retainer member (18) and said cap member (12) that said sealing member (56) is compressed when said cap member (12) is attached to said retainer member (18).
16. The medicament delivery device sub-assembly according to any one of the preceding clauses 1-9, wherein the distal pointed end (42) is arranged to penetrate a membrane arranged at the proximal end of the medicament container.
17. The medicament delivery device sub-assembly according to any one of the preceding clauses 1-15, wherein the hub (32) comprises on its outer circumferential surface a distal engagement member (36) and a proximal engagement member (46).
18. The medicament delivery device sub-assembly according to clause 17, wherein the retainer member (18) comprises a distal tubular part (20) having a container attachment member, a proximal tubular part (24) having an outer engagement member (44) on its outer circumferential surface, and a central passage (26) having opposite distal and proximal ends and an inner engagement member (30) on its inner circumferential surface adapted to receive the distal engagement member (36) of the hub (32).
19. The medicament delivery device sub-assembly according to clause 18, wherein the cap member (12) comprises on its inner circumferential surface a first engagement member (48) arranged to cooperate with the proximal engagement member (46) of the hub (32), and a second engagement member (50) arranged to cooperate with the outer engagement member (44) of the retainer member (18).
20. The medicament delivery device sub-assembly according to clause 19, wherein the first engagement member (48) of the cap member (12) is arranged to cooperate with the proximal engagement member (46) of the hub, such that the hub (32) is allowed to be axially displaceable but prevented to be rotated in relation to the cap member (12).
21. The medicament delivery device sub-assembly according to clause 20, wherein the second engagement member (50) of the cap member (12) is arranged to cooperate with the outer engagement member (44) of the retainer member (18) such that the cap member (12) is allowed to be axially displaceable in relation to the retainer member from a first position, in which the cap member (12) is prevented to be removed from the retainer member, to a second position, in which the cap member (12) is allowed to be removed from the retainer member.
22. The medicament delivery device sub-assembly according to clause 21, wherein the inner engagement member (30) of the retainer member (18) is arranged to cooperate with the distal engagement member (36) of the hub (32) such that the hub (32) is allowed to be coaxially displaceable in relation to the retainer member (18) from a disconnected position in which the distal engagement member (36) is positioned at the proximal end of the central passage (26) to a connected position in which the distal engagement member (36) is positioned at the distal end of the central passage (26).
23. The medicament delivery device sub-assembly according to clause 22, wherein the hub (32) is displaced from the disconnected to the connected position when the cap member (12) is displaced from the first to the second position.
24. The medicament delivery device sub-assembly according to clause 23, wherein the inner engagement member (30) of the retainer member (18) and the distal engagement member (36) of the hub (32) are guiding members.
25. The medicament delivery device sub-assembly according to clause 24, wherein the second engagement member (50) of the cap member (12) and the outer engagement member (44) of the retainer member (18) are guiding members.
26. The medicament delivery device sub-assembly according to clause 23, wherein the inner engagement member (30) of the retainer member (18) and the distal engagement member (36) of the hub (32) are threaded members.
27. The medicament delivery device sub-assembly according to clause 26, wherein the second engagement member (50) of the cap member (12) and the outer engagement member (44) of the retainer member (18) are threaded members.
28. Medicament delivery device comprising a medicament delivery device sub-assembly according to any one of the preceding clauses.
29. Medicament delivery device of clause 28, wherein the medicament delivery device is an autoinjector.

1-17. (canceled)

18: A medicament delivery device sub-assembly comprising an injection needle assembly, a cartridge and a seal, the injection needle assembly having opposite distal and proximal ends, the injection needle assembly comprising:
a retainer member configured to be connectable to the cartridge;
a hub configured to be coaxially displaceable within the retainer member and provided with an injection needle having proximal and distal pointed ends;
a cap member interactively connected to both the hub and to the retainer member; and
a sealing member arranged between said cap member and said retainer member for preventing contamination of said injection needle before use,
wherein the seal seals a gap between the injection needle assembly and the cartridge.

19: The medicament delivery device sub-assembly according to claim 18, wherein the seal is ring-shaped.

20: The medicament delivery device sub-assembly according to claim 18, wherein the seal comprises a central passage.

21: The medicament delivery device sub-assembly according to claim 18, wherein the seal is made of an elastic material.

22: The medicament delivery device sub-assembly according to claim 18, wherein the seal is made of a thermoplastic elastomer.

23: The medicament delivery device sub-assembly according to claim 18, wherein the seal is an O-ring.

24: The medicament delivery device sub-assembly according to claim 18, wherein the seal is a pad.

25: The medicament delivery device sub-assembly according to claim 18, comprising a transversal extending wall of the injection needle assembly, wherein the transversal extending wall is between the cartridge and the injection needle.

26: The medicament delivery device sub-assembly according to claim 18, wherein the seal is a distally extending extension of a transversal extending wall of the injection needle assembly, wherein the transversal extending wall is between the cartridge and the injection needle.

27: The medicament delivery device sub-assembly according to claim 18, wherein the seal is in the gap between the injection needle assembly and one or more of a cylindrical portion of the cartridge, a shoulder of the cartridge, a neck of the cartridge, a head of the cartridge, a membrane of the cartridge and a crimp of the cartridge.

28: The medicament delivery device sub-assembly according to claim 18, wherein the seal is in the gap between the cartridge and one or more of a transversal extending wall of the injection needle assembly and a distal tubular part of the injection needle assembly.

29: The medicament delivery device sub-assembly according to claim 18, wherein said sealing member comprises an elastic member.

30: The medicament delivery device sub-assembly according to claim 18, wherein said sealing member is generally ring-shaped.

31: The medicament delivery device sub-assembly according to claim 18, wherein the seal seals a gap between the retainer member of the injection needle assembly and the cartridge.

32: A medicament delivery device comprising a medicament delivery device sub-assembly according to claim 18.

33: The medicament delivery device of claim 32, wherein the medicament delivery device is an autoinjector.

34: A method of assembly of a medicament delivery device sub-assembly, the method comprising the steps of:
providing an injection needle assembly as described in claim 18;
providing a cartridge; and
assembling the cartridge and the injection needle assembly together with a seal in a sterile environment, so that the seal isolates a sterile volume between the cartridge and the injection needle assembly.

35: A sub-assembly for use in a medicament delivery device, where the sub-assembly comprises:
a retainer;
a cartridge containing medicament that is engaged with an inside portion of the retainer;
a first seal operatively engaged with both the inside portion of the retainer and with an outside portion of the cartridge;
a hub comprising a double ended injection needle, where the hub is operatively engaged with the retainer such that the hub moves axially relative to the retainer and the cartridge when a cap enclosing the injection needle is removed from the retainer; and
a second seal arranged between the cap and the retainer that prevents contamination of the injection needle before use when the cap is attached to the retainer.

36: The sub-assembly of claim 35, wherein the seal is ring-shaped, comprises a central passage and is made of an elastic material.

37: The sub-assembly of claim 36, wherein the seal is in the gap between the cartridge and one or more of a transversal extending wall of the retainer and a distal tubular part of the retainer, and
wherein the sealing member comprises an elastic member and is generally ring-shaped.
